(19) United States
(12) Patent Application Publication
(10) Pub. No.: US 2026/0144449 A1
(43) Pub. Date: May 28, 2026

(54) WEARABLE DEVICE WITH LIGHT SOURCE AND OPTICAL SENSOR

(71) Applicant: ROCKLEY PHOTONICS LIMITED, Altrincham (GB)

(72) Inventor: James McMILLAN, Santa Monica, CA (US)

(21) Appl. No.: 19/275,916

(22) Filed: Jul. 21, 2025

Related U.S. Application Data
(63) Continuation of application No. 18/984,845, filed on Dec. 17, 2024, now Pat. No. 12,396,648.
(60) Provisional application No. 63/726,157, filed on Nov. 27, 2024.

(51) Int. Cl. A61B 5/0295; A61B 5/021; A61B 5/026

(52) U.S. Cl. CPC A61B 5/0295; A61B 5/02141; A61B 5/0261

(57) ABSTRACT

A wearable device with a light source and an optical sensor. In some embodiments, a system includes a wearable device for being worn by a subject and for measuring a first biomarker of a blood vessel of the subject. The wearable device may include a first light source, and a plurality of optical detectors. The system may be configured: to perform a photoplethysmography measurement with each of the plurality of optical detectors, the first biomarker being a blood volume; or to perform a speckleplethysmography measurement with each of the plurality of optical detectors, the first biomarker being a blood flow velocity.

### CROSS-REFERENCE TO RELATED APPLICATION(S)

The present application is a continuation of U.S. patent application Ser. No. 18/984,845, filed Dec. 17, 2024, entitled “WEARABLE DEVICE WITH LIGHT SOURCE AND OPTICAL SENSOR”, which claims priority to and the benefit of U.S. Provisional Application No. 63/726,157, filed Nov. 27, 2024, entitled “WEARABLE DEVICE WITH LIGHT SOURCE AND OPTICAL SENSOR”; the entire contents of all of the documents identified in this paragraph are incorporated herein by reference.

### FIELD

One or more aspects of embodiments according to the present disclosure relate to biomarker monitoring, and more particularly to a system and method for measuring one or more biomarkers using one or more light sources and one or more optical sensors.

### BACKGROUND

Various cardiovascular biomarkers may be clinically significant, including, for example, the blood pressure of a subject.

It is with respect to this general technical environment that aspects of the present disclosure are related.

### SUMMARY

According to an embodiment of the present disclosure, there is provided a system, including: a wearable device for being worn by a subject and for measuring a first biomarker of a blood vessel of the subject, the wearable device including: a first light source, and a plurality of optical detectors, the system being configured: to perform a photoplethysmography measurement with each of the plurality of optical detectors, the first biomarker being a blood volume; or to perform a speckleplethysmography measurement with each of the plurality of optical detectors, the first biomarker being a blood flow velocity.

In some embodiments, the system is configured to read interleaved data from the plurality of optical detectors.

In some embodiments, the system is further configured: to read first interleaved data from a first subset of the plurality of optical detectors; and to read second interleaved data from a second subset of the plurality of optical detectors.

In some embodiments, the system is configured: to perform a first measurement, with a first optical detector of the plurality of optical detectors; to perform a second measurement, with a second optical detector of the plurality of optical detectors, the second optical detector being azimuthally separated from the first optical detector; and to determine that a measure of signal quality of the first measurement exceeds a measure of signal quality of the second measurement.

In some embodiments, the system is further configured: in response to the determining that the measure of signal quality of the first measurement exceeds the measure of signal quality of the second measurement, to perform a third measurement, with the first optical detector.

In some embodiments: the system is further configured: to read first interleaved data from a first subset of the plurality of optical detectors; and to read second interleaved data from a second subset of the plurality of optical detectors, wherein the first optical detector is an optical detector of the first subset, and the second optical detector is an optical detector of the second subset.

In some embodiments, the system is further configured: in response to the determining that the measure of signal quality of the first measurement exceeds the measure of signal quality of the second measurement, to prompt a user of the system to perform an azimuthal adjustment of the wearable device.

In some embodiments: the first biomarker is a blood flow velocity, and the plurality of optical detectors includes a first image sensor and a second image sensor azimuthally separated from the first image sensor.

In some embodiments, the first image sensor is a zero chief ray angle image sensor.

In some embodiments, an optical path between the blood vessel of the subject and the first image sensor includes no lenses.

In some embodiments, the wearable device further includes a receiving window, and the length of an optical path between the receiving window and an optical detector of the plurality of optical detectors is less than 15 mm.

In some embodiments, the wearable device further includes a receiving window, and a distance between the receiving window and an optical detector of the plurality of optical detectors is less than 5 mm.

In some embodiments: the wearable device further includes a second light source; and the system is further configured: to perform a photoplethysmography measurement with the second light source and an optical detector of the plurality of optical detectors; or to perform a speckleplethysmography measurement with the second light source and an optical detector of the plurality of optical detectors.

According to an embodiment of the present disclosure, there is provided a system, including: a wearable device for being worn by a subject and for measuring a first biomarker of a blood vessel of the subject, the wearable device including: a plurality of light sources, and a first optical detector, the system being configured: to perform a photoplethysmography measurement with each of the plurality of light sources, the first biomarker being a blood volume; or to perform a speckleplethysmography measurement with each of the plurality of light sources, the first biomarker being a blood flow velocity.

In some embodiments, the system is configured: to perform a first measurement, with a first light source of the plurality of light sources; to perform a second measurement, with a second light source of the plurality of light sources, the second light source being azimuthally separated from the first light source; and to determine that a measure of signal quality of the first measurement exceeds a measure of signal quality of the second measurement.

In some embodiments, the system is further configured: in response to the determining that the measure of signal quality of the first measurement exceeds the measure of signal quality of the second measurement, to perform a third measurement, with the first light source.

In some embodiments, the system is further configured: in response to the determining that the measure of signal quality of the first measurement exceeds the measure of signal quality of the second measurement, to prompt a user of the system to perform an azimuthal adjustment of the wearable device.

In some embodiments: the wearable device further includes a second optical detector; and the system is further configured: to perform a photoplethysmography measurement with a light source of the plurality of light sources and the second optical detector; or to perform a speckleplethysmography measurement with a light source of the plurality of light sources and the second optical detector.

### BRIEF DESCRIPTION OF THE DRAWINGS

These and other features and advantages of the present disclosure will be appreciated and understood with reference to the specification, claims, and appended drawings wherein:

FIG. 1A is a schematic drawing of a health sensor, according to an embodiment of the present disclosure;

FIG. 1B is a schematic drawing of a health sensor, according to an embodiment of the present disclosure;

FIG. 1C is a schematic drawing of a health sensor, according to an embodiment of the present disclosure;

FIG. 1D is a schematic drawing of a health sensor, according to an embodiment of the present disclosure;

FIG. 1E is a schematic drawing of a health sensor, according to an embodiment of the present disclosure;

FIG. 1F is a schematic drawing of a health sensor, according to an embodiment of the present disclosure;

FIG. 1G is a schematic drawing of a health sensor, according to an embodiment of the present disclosure;

FIG. 1H is a schematic drawing of a health sensor, according to an embodiment of the present disclosure;

FIG. 1I is a schematic drawing of a health sensor, according to an embodiment of the present disclosure;

FIG. 1J is a schematic drawing of a health sensor, according to an embodiment of the present disclosure;

FIG. 1K is a schematic drawing of a health sensor, according to an embodiment of the present disclosure;

FIG. 2A is a schematic drawing of an images sensor and processor interface, according to an embodiment of the present disclosure;

FIG. 2B is a schematic drawing of a plurality of images sensors and processor interfaces, according to an embodiment of the present disclosure;

FIG. 2C is a schematic drawing of a plurality of images sensors and processor interfaces, according to an embodiment of the present disclosure;

FIG. 2D is a schematic drawing of a plurality of images sensors and processor interfaces, according to an embodiment of the present disclosure;

FIG. 3A is a schematic drawing of a sensor with four image sensors, over an artery, according to an embodiment of the present disclosure;

FIG. 3B is a graph of data corresponding to the configuration of FIG. 3A, according to an embodiment of the present disclosure;

FIG. 3C is a schematic drawing of a sensor with four image sensors, over an artery, according to an embodiment of the present disclosure; and

FIG. 3D is a graph of data corresponding to the configuration of FIG. 3C, according to an embodiment of the present disclosure; and

FIG. 4 is a flow chart, according to an embodiment of the present disclosure.

### DETAILED DESCRIPTION

The detailed description set forth below in connection with the appended drawings is intended as a description of exemplary embodiments of a wearable device with a light source and an optical sensor provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

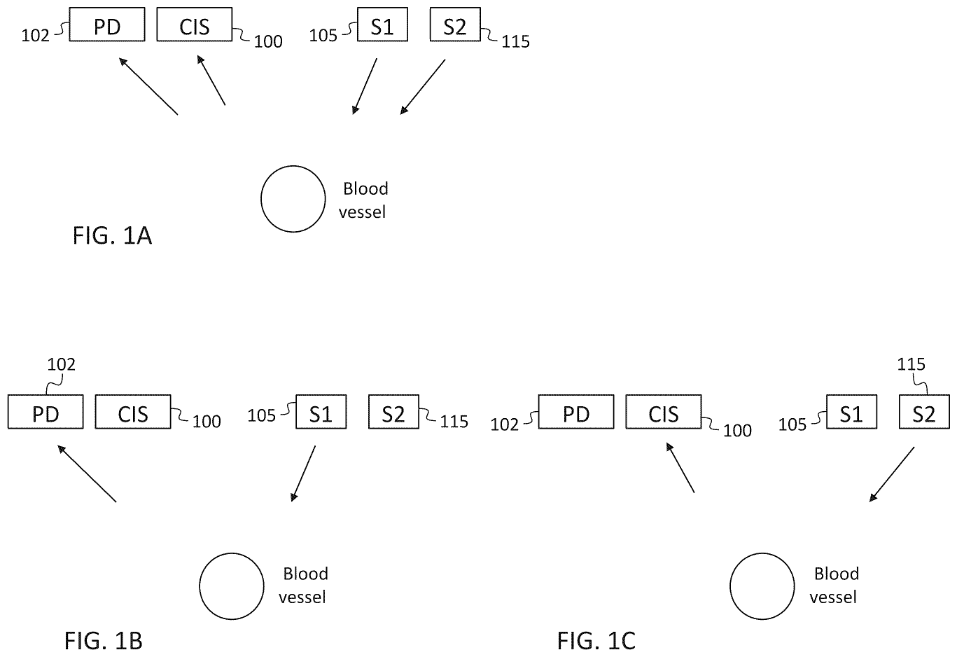

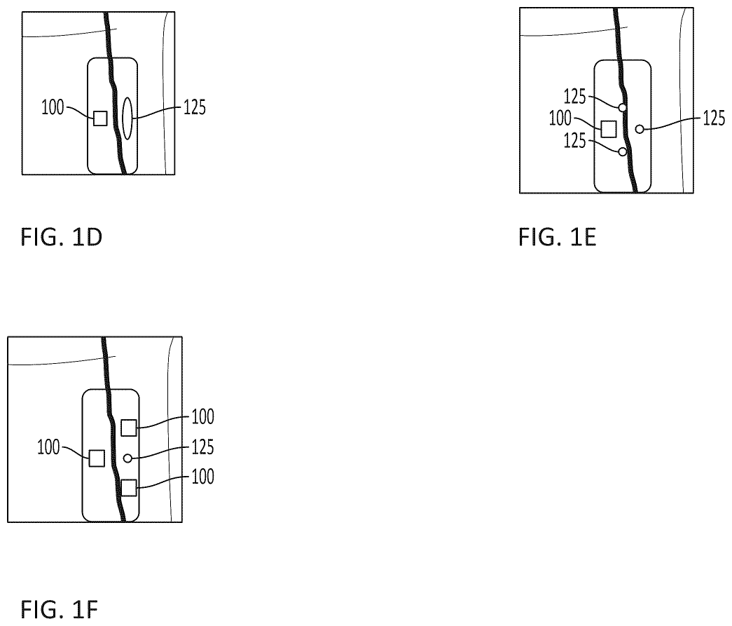

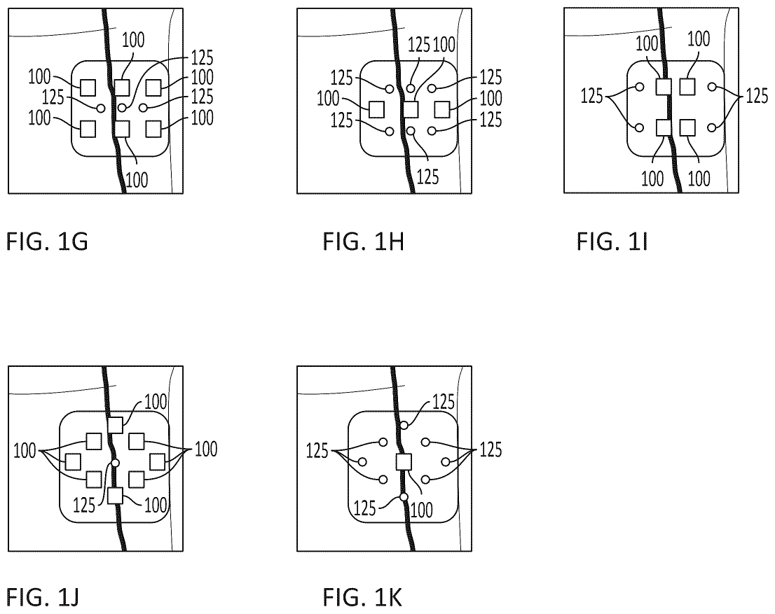

Referring to FIG. 1A, a health sensor (e.g., a cuffless blood pressure sensor) may include one or more light sources 110 for illuminating one or more blood vessels in a patient, or “subject”, and optical detectors 120 (e.g., a photodiode (PD) 102 or an image sensor 100 (e.g., a CMOS image sensor (CIS)) for receiving light scattered from the blood vessel (e.g., from blood inside the blood vessel). The health sensor may include (i) a photoplethysmography (PPG) sensor including a source of incoherent light 105 (e.g., S1, in FIG. 1A) and a photodetector (e.g., a single-pixel photodetector such as a photodiode 102 (PD)) for measuring the instantaneous blood volume in the blood vessel, and (ii) a speckleplethysmography (SPG) sensor (including a source of coherent light such as a laser 115 (e.g., S2, in FIG. 1A) and an image sensor 100 (e.g., a CMOS image sensor (CIS) or a charge-coupled device (CCD)) for measuring the instantaneous blood velocity in the blood vessel. Each of the instantaneous blood volume in the blood vessel, and the instantaneous blood velocity may vary with the cardiac cycle; blood pressure (both the systolic blood pressure and the diastolic blood pressure) may be inferred from a time history (or “time series”) of instantaneous blood volume measurements and a time history of instantaneous blood velocity measurements.

FIG. 1B shows the performing of a photoplethysmography measurement (with the blood vessel illuminated by the source of incoherent light, and the photodiode 102 (PD)) being used to detect the light scattering from the blood vessel into the photodiode 102. FIG. 1C shows the performing of a speckleplethysmography measurement (with the blood vessel illuminated by the source of coherent light S2, and the image sensor 100 (labeled CIS) being used to detect the light scattering from the blood vessel into the image sensor 100. When a speckleplethysmography measurement is performed, the measured speckle contrast (which is a measurement that is time-averaged over the exposure time of the image sensor 100) may decrease with increasing blood flow velocity (which may cause the speckle pattern on the image sensor 100 to change more rapidly); as such, the measured speckle contrast may be used as an indication of blood flow velocity.

Each image (or frame) generated by the image sensor 100 may be processed to calculate a data point of a time series representing a biomarker. One such biomarker may be, as mentioned above, a value that is representative of flow of blood in an artery. In such a case the alignment of the light source 110 and image sensor 100 may be such that the light scattered by the flowing blood in the artery is able to be captured by the imaging device.

For the case in which a health sensor based on laser speckle imaging (e.g., a health sensor based on speckleplethysmography) is used in a wearable device to measure the blood flow in a particular anatomical feature such as an artery, for example the radial artery at the wrist, a need to align the sensor with this anatomical feature may make the sensor performance highly dependent on device placement. The small radial artery diameter (2-3 mm) combined with the limited sensing volume offered by a sensor consisting of a single light source 110 and single image sensor 100 may contribute to this dependency on device placement.

Placing a wearable device over a non-visible blood vessel such as the radial artery on the volar side of the wrist may be challenging, especially when the body of the wearable device itself obstructs the view of skin when placed in the general area of the artery. This limits the user's ability to align the emission and detection windows of the wearable device with a location on the skin that might have been identified pre-placement via ultrasound imaging or palpitation. This last issue may be mitigated somewhat using datums on the body of the wearable device that may be positioned relative to landmarks of the subject's anatomy for alignment to the wearable device's optimal sensing point. A multi-step placement process involving first locating the blood vessel (e.g., by palpitation) relative to landmarks of the subject and then placing the wearable device at a location defined by the landmarks may be challenging for an untrained user, however. As used herein, the subject is a person wearing the wearable device, and the “user” is a person operating the wearable device. The user may be the subject or another person, e.g., a clinician.

Various aspects of the design of the wearable device may affect sensitivity to alignment. FIG. 1D shows an illumination shape in which the laser beam 125 is highly elliptical, so as to illuminate more tissue while not greatly impacting source-detector separation. Such a configuration may impact signal-to-noise ratio (SNR) performance by the addition of light not travelling through the radial artery, however. FIG. 1E shows a configuration with multiple emitters (e.g., multiple lasers 115) illuminating, with multiple respective laser beams 125, the tissue of the subject, and the artery. In such an embodiment, the lasers 115 may be turned on one at a time, to identify the laser 115 associated with the best signal-to-noise ratio performance. FIG. 1F shows an embodiment with multiple optical detectors 120. For example, three image sensors 100 may be operated in “multi-sensor” mode where frames are captured sequentially from each sensor. The effective frame rate may be divided by 3 (in the case of using 3 image sensors 100). In some such embodiments, the image sensor 100 with the best performance (which may depend on the location, relative to the light source 110 and the artery, of each of the image sensors 100) may be identified and the wearable device may then transition to using only this image sensor 100, at full frame rate. FIGS. 1G-11 show embodiments with multiple light sources 110 (e.g., multiple lasers 115 illuminating the tissue of the subject with multiple respective laser beams 125) and multiple optical detectors 120. FIGS. 1J and 1K show, respectively, an embodiment with multiple optical detectors 120 and one light source 110, and with multiple light sources 110 and one optical detector 120.

As mentioned above, to reduce the positional sensitivity of a sensor the number of transmitting elements or the number of receiving elements may be increased. In the case of a laser-based speckle imaging sensor (e.g., in the case of a speckleplethysmography sensor) this may be accomplished by increasing the number of lasers 115 or the number of image sensors 100 (or both). If multiple lasers 115 (that are not phase coherent with each other) are used, then only one laser 115 may be turned on at a time, to avoid the reduction in speckle contrast that may otherwise occur when light from several lasers 115 is received at an image sensor 100. For a convex, generally cylindrical part of the subject (such as a finger, a limb, the neck, or the torso of the subject) (which may be referred to as a “convexity” of the subject) with a blood vessel (e.g., the radial artery) extending generally parallel to, and offset from a central axis of, the part (as, for example, the radial artery at the wrist extends generally parallel to the arm and near the skin) the degree of freedom in which accurate placement may be important may be the “azimuthal” position of the sensor (which for a band securing the wearable device to the wrist may be adjusted by rotating the band (along with the wearable device) around the wrist (about the longitudinal axis of the forearm)).

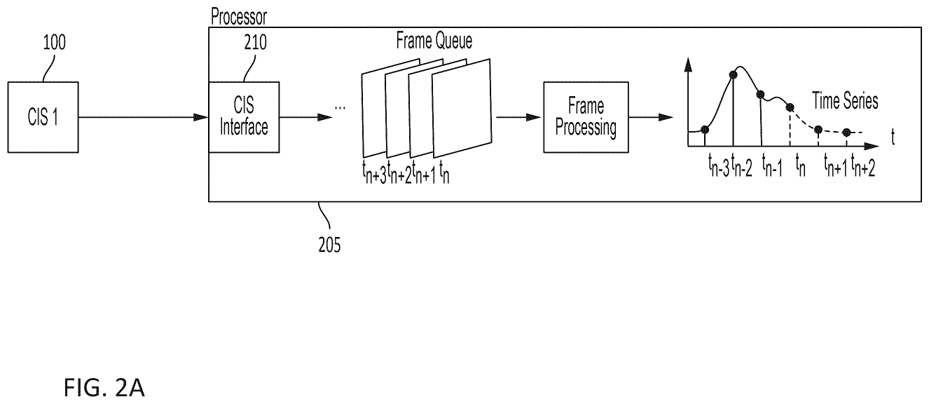

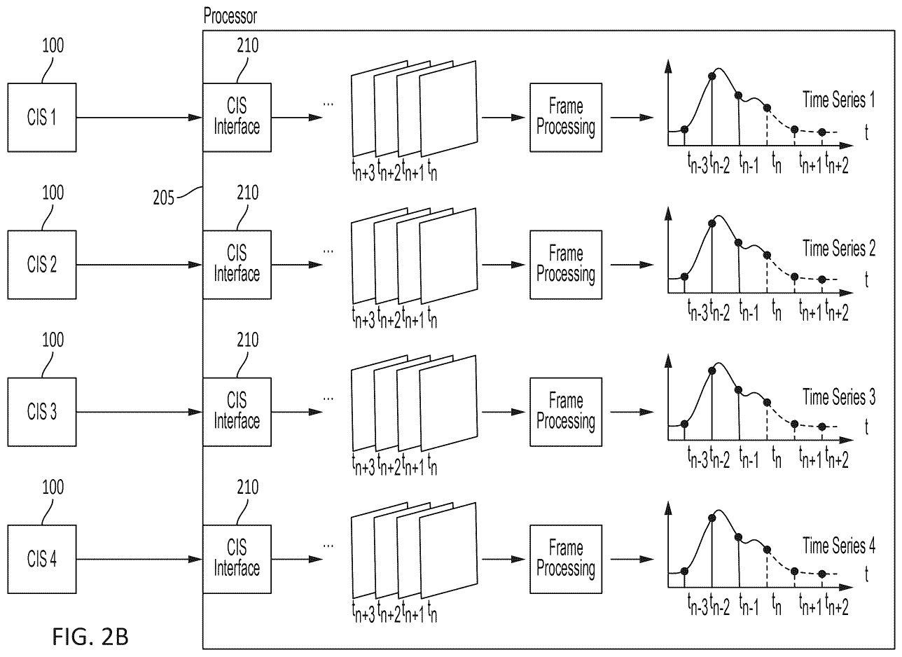

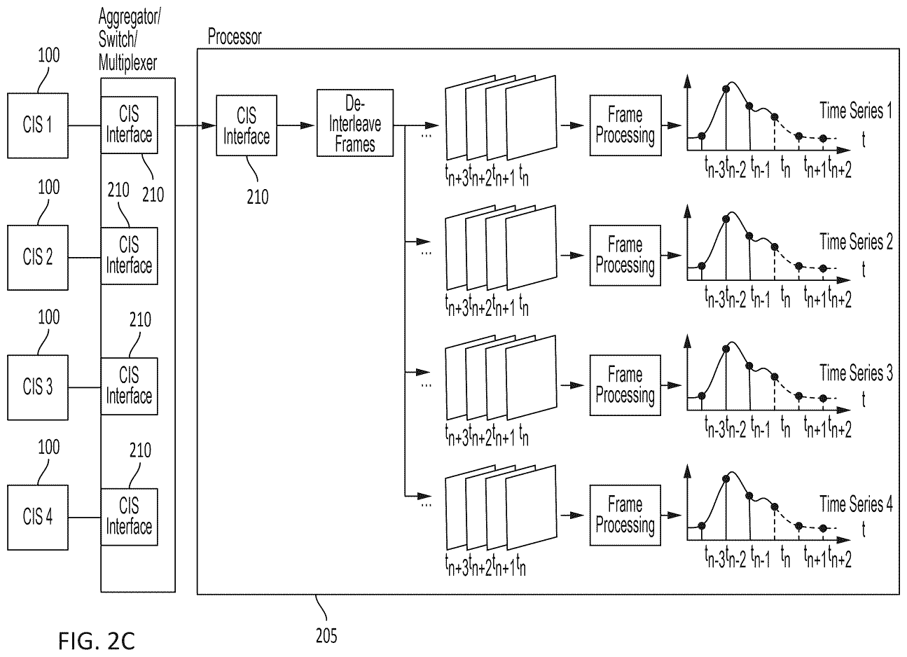

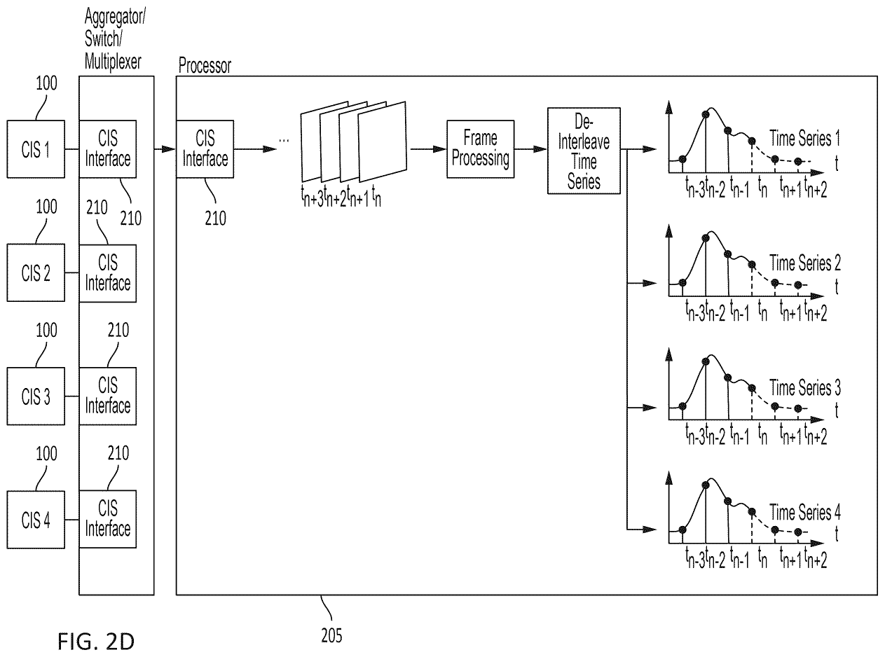

Using multiple image sensors 100 offers the capability to detect over a broader physical space and cover regions where the scattered light would be generally lost when a single sensor is employed. FIG. 2A shows a simplified signal chain of the receiving elements of the wearable device. An image sensor 100 transmits image data (frames) to a processor 205 (e.g., a processing circuit) (such as a microcontroller (MCU) or field programmable gate array (FPGA)). The processor 205 receives frame data from the image sensor 100 via an interface 210 (such as a parallel bus interface or a serial interface such as the Camera Serial Interface (CSI) (promulgated by the Mobile Industry Processor Interface (MIPI) Alliance). The frames may be queued before frame processing. The queue may have a length of zero in which case frame processing occurs immediately. Frame processing extracts information from each frame received, resulting in value representative of the state of the speckle in the imaged frame captured at a time t. This value may be, for example, a measured speckle contrast. The image sensor 100 operates at a frame rate which may define the sample rate of the time series.

In some embodiments, multiple image sensors 100 are operated in parallel as illustrated in FIG. 2B. In the example shown, four image sensors 100 are shown but the design may be employed with more or fewer image sensors 100. The processor 205 in this embodiment need not be a single processor 205 but may be a distributed processor 205, including a plurality of processors 205.

In some embodiments, multiple image sensors 100 share a communication bus and an interface 210 with the processor 205, as shown in FIG. 2C. The processor 205 may have a limited number of sensor interfaces 210. An aggregator or switch or multiplexer (external to the processor 205) may be connected to the image sensors 100, and, in operation, may interleave their frame streams and transmit the resulting composite stream to the processor 205 through a single CIS interface 210. In some embodiments, a switch or multiplexer is employed to switch between generated frames, so the processor 205 receives an interleaved sequence. A switch may also be employed in a non-interleaved or non-simultaneous sampling embodiment. In such an embodiment, each image sensor 100 may be monitored for a few seconds sequentially to assess signal quality. The optimal image sensor 100 may then be identified. In this embodiment the image sensors 100 may capture frames at a simultaneous point in time at a frame rate F and transmit those frames to the processor 205 at rate NF (where N is the number of image sensors 100; e.g., N=4 in the embodiment of FIG. 2C). In some embodiments, the image sensors 100 instead capture frames sequentially, so that the rate at which each sensor runs is F/N, and the rate at which the frames are sent to the processor 205 is equal to F. In both cases the captured frames may be de-interleaved, e.g., in the processor 205 (or in a separate de-interleaving circuit) to separate into N respective streams the frames from the N image sensors 100. The processor 205 may then process the frames provided by the image sensors 100 in parallel, as illustrated in FIG. 2C.

In some embodiments, e.g., if the processor 205 lacks sufficient processing resources to process the frames in parallel, the frames are processed one at a time (e.g., in series) in the processor 205 (instead of being processed in parallel). In such an implementation, an example of which is shown in FIG. 2D, the frames in the stream provided by the image sensors 100 are processed sequentially and the corresponding data points are de-interleaved to produce a corresponding respective time series for each of the image sensors 100.

In some embodiments, the image sensors 100 are grouped into subsets, and one subset of image sensors 100 is read out at a time, in parallel or in an interleaved manner. For example, after one or more frames have been read from each of a first subset of image sensors 100, the system may switch to reading out a second subset of the image sensors 100, e.g., in an interleaved manner. Once one or more frames have been read from each of the second subset of image sensors 100, the system may switch to a third subset of the image sensors 100, and so on, or it may switch back to the first subset of the image sensors 100.

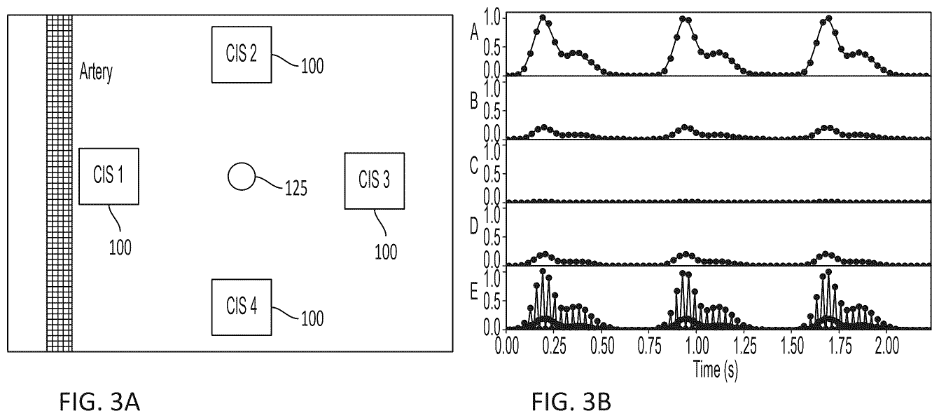

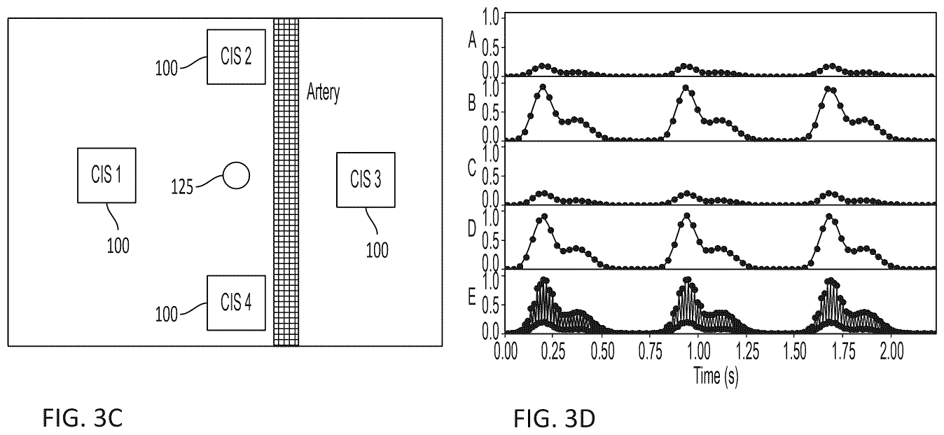

FIG. 3A shows (i) a speckleplethysmography sensor including four image sensors 100 and a single light source 110, and (ii) an artery that is the target of the speckleplethysmography sensor. The resulting representative time series from CIS 1 through 4 are shown in plot A though D respectively, of FIG. 3B; plot E of FIG. 3B shows the composite time series before de-interleaving.

FIG. 3C shows the speckleplethysmography sensor of FIG. 3A after the position of the wearable device with respect to the artery has changed (e.g., as a result of a user's moving of the wearable device). The effect on the resulting time series may be seen in the plots in FIG. 3D (in which representative time series from CIS 1 through 4 are shown in plot A though D respectively, and plot E shows the composite time series before de-interleaving); the magnitude of the time series data collected by CIS 2 and 4 is increased in comparison with the times series data corresponding to the position of FIG. 3A.

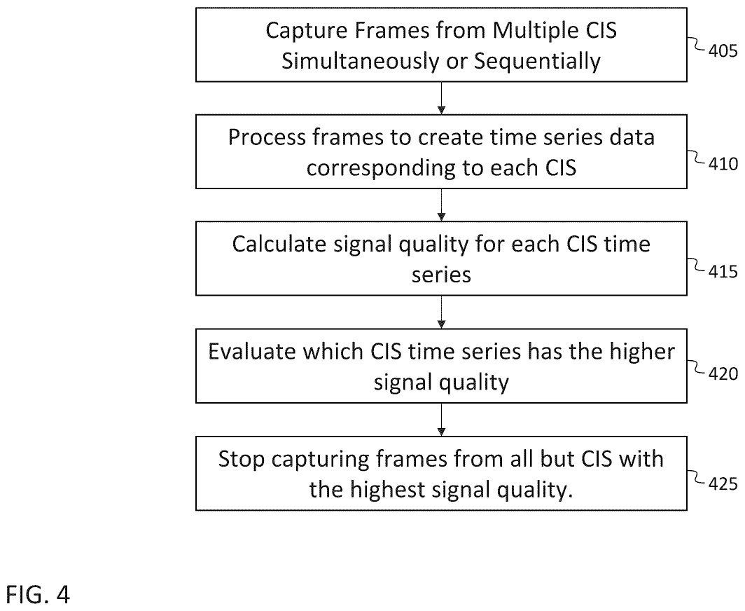

In some embodiments, a speckleplethysmography sensor utilizing multiple image sensors 100 may at startup, or periodically, be operated with all of the image sensors 100 and may then transition to a mode in which only a single image sensor 100 operates, e.g., to conserve power, or to permit the capturing and processing of images at a higher rate. The single image sensor 100 used after the transition may be the image sensor 100 generating the highest signal quality before the transition, where signal quality may be measured by a suitable algorithm (e.g. maximum peak-to-peak amplitude). FIG. 4 shows a flow chart corresponding to such a method. In the method of FIG. 4, frames are captured simultaneously from multiple image sensors 100, at 405; the frames are processed, at 410, to create time series data corresponding to each image sensor 100; the signal quality for each such time series is calculated, at 415; the image sensor 100 producing signals with the highest signal quality is identified, at 420, and the system switches, at 425, to a mode in which it uses only this identified image sensor 100. A transition back to a mode in which all of the image sensors 100 are used may be triggered by an external stimulus (e.g., an accelerometer reading exceeding a threshold) or by a real-time evaluation of the sensor time series data (e.g., peak-to-peak speckle contrast amplitude being below a threshold).

In some embodiments, a difference in signal quality between two image sensors 100 may cause the wearable device to prompt a user to perform an azimuthal adjustment of the wearable device, so as to improve the positioning of the wearable device. For example, in the situation illustrated in FIG. 3A, the system may infer, from the higher signal quality at CIS 1, that an azimuthal displacement of the wearable device, so that the position of the artery is nearer the center of the constellation of image sensors 100, may improve the signal quality at one or more of the image sensors 100, and the wearable device may prompt the user accordingly.

In some embodiments, multiple photodiodes 102, or multiple light sources 110 (e.g., multiple coherent light sources or multiple incoherent light sources) may be used in an analogous manner, to reduce the sensitivity to placement of the wearable device, or to generate information based on which a user may be prompted to adjust the azimuthal position of the wearable device.

In some embodiments with multiple image sensors 100, each image sensor 100 may receive light through a respective separate window in the wearable device (which may be shared with a respective photodiode 102), and a separate window may be used for each light source 110, or for all of the light sources 110.

An image sensor 100 designed for imaging applications may include a micro-lens array that includes one micro-lens for each pixel in the image sensor 100. Each micro-lens may concentrate the received light on the light-sensitive portion of the corresponding pixel (in some devices part of the area of the pixels is allocated to a control and readout circuit and is not light-sensitive). Such a microlens may be offset from the center of the light-sensitive portion of the corresponding pixel if the chief ray angle for the pixel, in an optical system for which the image sensor 100 is intended, is not zero, i.e., if the chief ray corresponding to the pixel is not perpendicular to the plane of the image sensor 100. This may be because the pixel is not near the center of the array (e.g., because the pixel is near the edge of the array). The offsetting of microlenses from the light-sensitive portion of the corresponding pixels may result in vignetting (lower exposure at and near the edges of the image sensor 100) in applications (such as the optodes described herein) in which the illumination is generally diffuse Lambertian illumination, which is approximately isotropic over a large solid angle, because in such a configuration a greater proportion of the received light may miss the light-sensitive portion of the corresponding pixel if the microlens is offset from the light-sensitive portion of the corresponding pixel. As such, in some embodiments, an image sensor 100 that does not include a micro-lens array, or an image sensor 100 that includes a micro-lens array and that is a zero chief ray angle (CRA) image sensor (e.g., an image sensor 100 in which each micro-lens is aligned with (not offset from) the light-sensitive portion of the corresponding pixel) may be used. As used herein, a zero chief ray angle sensor is a sensor (i) that does not include a microlens array or (ii) that includes a micro-lens array in which each chief ray angle is small (e.g., less than 5 degrees) (where the chief ray is a ray that passes through the center of the aperture and ends at the center of the light-sensitive area of the pixel, and the chief ray angle is the angle between the chief ray and a line perpendicular to the plane of the detector 120).

The distance from the sample (e.g., from the skin of the subject) to the image sensor 100 may be less than 5 mm (in a straight line) or less than 15 mm along the optical path (the latter distance being greater than the former if, for example, a folding mirror is used to cause the light from the sample to propagate parallel to the surface of the sample for some distance before reaching the image sensor 100).

The wearable device may be coupled to various other devices and systems which may perform functions of data analysis, data storage, and user interaction. For example, the wearable device may be connected (e.g., via a wireless connection) to a mobile device (such as a mobile telephone or portable (e.g., laptop) computer) and a portion of the data analysis used to convert the signals received from the optical sensors to one or more biomarkers (e.g., blood flow velocity, blood volume, or blood pressure) may be performed in the mobile device. The mobile device may also be connected to the cloud (e.g., to one or more servers connected to the Internet) and data processing and storage may be performed on the cloud. As another example, if an adjustment is to be made (e.g., an adjustment to the azimuthal position of the wearable device), the wearable device may cause the mobile device to give the user a suitable corresponding prompt.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

It will be understood that when an element is referred to as being “directly connected” or “directly coupled” to another element, there are no intervening elements present. As used herein, “generally connected” means connected by an electrical path that may contain arbitrary intervening elements, including intervening elements the presence of which qualitatively changes the behavior of the circuit. As used herein, “connected” means (i) “directly connected” or (ii) connected with intervening elements, the intervening elements being ones (e.g., low-value resistors or inductors, or short sections of transmission line) that do not qualitatively affect the behavior of the circuit.

Although exemplary embodiments of a wearable device with a light source and an optical sensor have been specifically described and illustrated herein, many modifications and variations will be apparent to those skilled in the art. Accordingly, it is to be understood that a wearable device with a light source and an optical sensor constructed according to principles of this disclosure may be embodied other than as specifically described herein. The invention is also defined in the following claims, and equivalents thereof.

1. A system, comprising:
a wearable device for being worn by a subject and for measuring a first biomarker of a blood vessel of the subject,
the wearable device comprising:
a first light source, and
a plurality of optical detectors,
the system being configured:
to perform a photoplethysmography measurement with each of the plurality of optical detectors, the first biomarker being a blood volume; or
to perform a speckleplethysmography measurement with each of the plurality of optical detectors, the first biomarker being a blood flow velocity.

2. The system of claim 1, wherein each of the plurality of optical detectors is configured to be illuminated by the first light source through the blood vessel.

3. The system of claim 2, wherein the plurality of optical detectors comprises a plurality of image sensors, and the system is configured to perform a speckleplethysmography measurement with each of the plurality of image sensors in response to the image sensor being illuminated by the first light source through the blood vessel.

4. The system of claim 1, wherein the system is configured to read interleaved data from the plurality of optical detectors.

5. The system of claim 4, wherein the system is further configured:
to read first interleaved data from a first subset of the plurality of optical detectors; and
to read second interleaved data from a second subset of the plurality of optical detectors.

6. The system of claim 1, wherein the system is configured:
to perform a first measurement, with a first optical detector of the plurality of optical detectors;
to perform a second measurement, with a second optical detector of the plurality of optical detectors, the second optical detector being azimuthally separated from the first optical detector; and
to determine that a measure of signal quality of the first measurement exceeds a measure of signal quality of the second measurement.

7. The system of claim 6, wherein the system is further configured:
in response to the determining that the measure of signal quality of the first measurement exceeds the measure of signal quality of the second measurement, to prompt a user of the system to perform an azimuthal adjustment of the wearable device.

8. The system of claim 6, wherein the system is further configured:
in response to the determining that the measure of signal quality of the first measurement exceeds the measure of signal quality of the second measurement, to perform a third measurement, with the first optical detector.

9. The system of claim 8, wherein:
the system is further configured:
to read first interleaved data from a first subset of the plurality of optical detectors; and
to read second interleaved data from a second subset of the plurality of optical detectors,
wherein the first optical detector is an optical detector of the first subset, and the second optical detector is an optical detector of the second subset.

10. The system of claim 1, wherein:
the first biomarker is a blood flow velocity, and
the plurality of optical detectors comprises a first image sensor and a second image sensor azimuthally separated from the first image sensor.

11. The system of claim 10, wherein the first image sensor is a zero chief ray angle image sensor.

12. The system of claim 11, wherein an optical path between the blood vessel of the subject and the first image sensor includes no lenses.

13. The system of claim 1, wherein:
the wearable device further comprises a second light source; and
the system is further configured:
to perform a photoplethysmography measurement with the second light source and an optical detector of the plurality of optical detectors; or
to perform a speckleplethysmography measurement with the second light source and an optical detector of the plurality of optical detectors.

14. A system, comprising:
a wearable device for being worn by a subject and for measuring a first biomarker of a blood vessel of the subject,
the wearable device comprising:
a plurality of light sources, and
a first optical detector,
the system being configured:
to perform a photoplethysmography measurement with each of the plurality of light sources, the first biomarker being a blood volume; or
to perform a speckleplethysmography measurement with each of the plurality of light sources, the first biomarker being a blood flow velocity.

15. The system of claim 14, wherein the first optical detector comprises an image sensor configured to be illuminated by each of the plurality of light sources through the blood vessel.

16. The system of claim 14, wherein the wearable device further comprises a receiving window, and the length of an optical path between the receiving window and the first optical detector is less than 15 mm.

17. The system of claim 14, wherein the system is configured:
to perform a first measurement, with a first light source of the plurality of light sources;
to perform a second measurement, with a second light source of the plurality of light sources, the second light source being azimuthally separated from the first light source; and
to determine that a measure of signal quality of the first measurement exceeds a measure of signal quality of the second measurement.

18. The system of claim 17, wherein the system is further configured:
in response to the determining that the measure of signal quality of the first measurement exceeds the measure of signal quality of the second measurement, to perform a third measurement, with the first light source.

19. The system of claim 17, wherein the system is further configured:
in response to the determining that the measure of signal quality of the first measurement exceeds the measure of signal quality of the second measurement, to prompt a user of the system to perform an azimuthal adjustment of the wearable device.

20. The system of claim 14, wherein:
the wearable device further comprises a second optical detector; and
the system is further configured:
to perform a photoplethysmography measurement with a light source of the plurality of light sources and the second optical detector; or
to perform a speckleplethysmography measurement with a light source of the plurality of light sources and the second optical detector.
